(19) United States
(12) Patent Application Publication
(10) Pub. No.: US 2023/0190835 A1
(43) Pub. Date: Jun. 22, 2023

(54) PROBIOTIC BACTERIA FOR TREATING ALZHEIMER'S DISEASE

(71) Applicants: Frederikus Gerardus Josephus Maria VAN DE WIEL, Waalwijk (NL); RRW B.V., Waalwijk (NL)

(72) Inventors: Frederikus Gerardus Josephus Maria VAN DE WIEL, Waalwijk (NL); Marjolein WILDWATER, Heerhugowaard (NL)

(21) Appl. No.: 17/926,164

(22) PCT Filed: May 18, 2021
(86) PCT No.: PCT/EP2021/063116

§ 371 Date: Nov. 18, 2022

(30) Foreign Application Priority Data
May 18, 2020 (DE) 10 2020 002 949.3

(51) Int. Cl. A61K 35/747; A61P 25/28; A61K 31/05; A61K 31/352; A61K 31/12

(52) U.S. Cl. CPC A61K 35/747; A61P 25/28; A61K 31/05; A61K 31/352; A61K 31/12

(57) ABSTRACT

The present invention relates to the treatment or prevention of amyloidosis by means of probiotic bacteria, in particular in combination with herbal active agents. In particular embodiments, probiotic bacteria are used together with a curcuminoid and/or a cannabinoid to treat or prevent Alzheimer's disease.

### CROSS-REFERENCE TO RELATED APPLICATIONS

This application is a 35 U.S.C. § 371 national stage filing of International Application No. PCT/EP2021/063116 filed on May 18, 2021, which claims the benefit of German Patent Application No. 10 2020 002 949.3, filed on May 18, 2020. The entire contents of each of these applications are incorporated herein by reference in their entirety.

### FIELD OF THE INVENTION

The present invention relates to the technical field of treatment of amyloidoses such as Alzheimer's disease. In particular, the invention relates to therapies for the treatment of Alzheimer's disease with probiotic bacteria preferably in combination with cannabinoids and/or curcuminoids.

### TECHNICAL BACKGROUND

One of the most common forms of neurodegenerative disease is Alzheimer's disease, which is also known synonymously as Morbus Alzheimer. Alzheimer's disease occurs particularly in older people beyond the age of 65, although the corresponding precursor forms or preclinical or clinical precursors may occur much earlier.

The prevalence of neurodegenerative diseases, and Alzheimer's disease in particular, is strongly increasing and its prevalence is steadily increasing worldwide, which will place severe strains on public health systems in the future. Alzheimer's disease is the most widespread form of dementia. Alzheimer's disease is associated with progressive deterioration of cognitive performance over time, usually accompanied by a decline in daily activities, behavioral abnormalities, and neuropsychological symptoms.

Characteristic of Alzheimer's disease are the plaques that form in the affected person's brain, which essentially have misfolded amyloid-β peptides, and the neurofibrils that accumulate in the neurons. The intracellularly located neurofibril bundles are substantially composed of the tau protein, which aggregates into fibrils when it is excessively phosphorylated. The amyloid-β peptides, which are also synonymously referred to as Aβ proteins, are formed from the so-called amyloid precursor protein (APP). APP is a type I transmembrane protein that is cleaved by certain proteinases. Cleavage of APP results in the release of Aβ-peptides from the precursor protein. Aβ-peptides are generally 37 to 42 amino acids in length and are associated with the pathological formation of Alzheimer's disease.

Deposits of proteins such as the amyloid protein or the tau protein also play a crucial role in other diseases. The protein deposits (amyloids) cause damage to cells and tissues, with the clinical picture being defined according to the location and type of deposition.

The objective of the present invention is to provide an improved prevention or treatment of these amyloidoses, in particular Alzheimer's disease.

### SUMMARY OF THE INVENTION

The invention is based on the finding that the therapeutic effect of various active agents in the treatment of amyloidoses such as Alzheimer's disease can be enhanced by combining them with probiotic bacteria. Synergistic effects of these combination therapies have been demonstrated in an animal model. In addition, the probiotic bacteria alone also show therapeutic activity.

Based on this finding, in a first aspect, the present invention provides the use of a probiotic bacterium for the manufacture of a medicament for the treatment or prevention of amyloidosis.

In a second aspect, the present invention relates to the use of a mixture of a probiotic bacterium and an active agent that is therapeutically effective in the treatment or prevention of amyloidosis for the manufacture of a medicament for the treatment or prevention of amyloidosis.

In preferred embodiments, the active agent is a cannabinoid, a curcuminoid, or a mixture thereof.

Other purposes, features, advantages and aspects of the present application will be apparent to those skilled in the art from the following description and the appended claims. Various changes and modifications in the spirit and scope of the disclosed invention will be apparent to those skilled in the art from the following disclosure.

### DETAILED DESCRIPTION OF THE INVENTION

Different bacterial species, such as probiotic bacteria in particular, showed beneficial effects in the treatment and prevention of amyloidosis in an animal model. With the additional administration of other active agents for the treatment of amyloidosis, in particular cannabinoid compounds such as cannabidiol (CBD) and delta-9-tetrahydrocannabinolic acid (THCA), and curcuminoid compounds such as curcumin, these effects were surprisingly significantly increased. Probiotic bacteria alone or in combination with a cannabinoid and/or a curcuminoid can thus be used in the treatment or prevention of amyloidoses such as Alzheimer's disease, obtaining a significantly improved therapeutic effect by the described combination.

With this in mind, in a first aspect, the present invention provides the use of a probiotic bacterium for the manufacture of a medicament for the treatment or prevention of amyloidosis.

The probiotic bacterium can be any probiotic bacterium as well as mixtures of different probiotic bacteria. A “bacterium” as used in the present invention generally refers to a plurality of bacterial cells rather than a single bacterial cell. In certain embodiments, the term “bacterium” refers to a plurality of bacterial cells of the same genus, species, or strain. Probiotic bacteria, in particular, are live bacteria that produce health benefits when administered in adequate amounts.

In particular embodiments, the probiotic bacterium is selected from the group consisting of bacteria of the genus Lactobacillus, bacteria of the genus Streptococcus, bacteria of the genus Bifidobacterium, bacteria of the genus Enterococcus, bacteria of the genus Pseudomonas, and mixtures of bacteria containing bacteria of one or more of these genera. For example, the probiotic bacterium may be selected from the group consisting of bacteria of the genus Lactobacillus, bacteria of the genus Streptococcus, bacteria of the genus Bifidobacterium, and bacteria of the genus Enterococcus. In particular embodiments, the probiotic bacterium is a lactic acid bacterium. In particular, the probiotic bacterium may be a bacterium of the genus Lactobacillus or a bacterial mixture containing a bacterium of the genus Lactobacillus.

In certain embodiments, the probiotic bacterium is selected from the group consisting of Enterococcus faecium, Bifidobacterium longum, Streptococcus thermophilus, Lactobacillus acidophilus, Lactobacillus plantarum, Lactobacillus rhamnosus, Bifidobacterium breve, Pseudomonas fluorescens, and mixtures of bacteria containing bacteria of one or more of these species. For example, the probiotic bacterium may be selected from the group consisting of bacteria of the species Enterococcus faecium, Bifidobacterium longum, Streptococcus thermophilus, Lactobacillus acidophilus, Lactobacillus plantarum, Lactobacillus rhamnosus, and Bifidobacterium breve. In particular embodiments, the probiotic bacterium is a bacterium of the species Lactobacillus plantarum or Lactobacillus rhamnosus or a bacterial mixture containing a bacterium of the species Lactobacillus plantarum and/or Lactobacillus rhamnosus. In other embodiments, the probiotic bacterium is a bacterium of the species Pseudomonas fluorescens.

In certain embodiments, the probiotic bacterium is administered in combination with an active agent that is therapeutically effective in treating or preventing amyloidosis. In these embodiments, the probiotic bacterium and the active agent may be present together in a mixture and administered to the patient. Alternatively, the probiotic bacterium and the active agent may each be present in a separate composition and administered to the patient. Several different probiotic bacteria and/or several different active agents may also be administered, wherein they may be administered together in one composition or separately in several separate compositions. Administration of several compositions may be simultaneous or sequential. Consecutive administration is particularly performed on the same day, preferably within a period of 4 hours, 2 hours, or more preferably one hour.

In a second aspect, the present invention relates also to the use of a mixture of a probiotic bacterium and an active agent that is therapeutically effective in the treatment or prevention of amyloidosis for the manufacture of a medicament for the treatment or prevention of amyloidosis.

The active agent that is therapeutically effective in the treatment or prevention of amyloidosis may be any therapeutically effective active agent, particularly an active agent that can be used to treat and/or prevent Alzheimer's disease. In particular embodiments, the active agent is a plant-derived active agent or an active agent obtainable from plants. In particular, the active agent can also be a mixture of different substances. In certain embodiments, the active agent is a cannabinoid, a curcuminoid, or a mixture thereof.

In particular, a therapeutic composition is a composition suitable for administration to a human or animal. A therapeutic composition consists in particular of pharmaceutically acceptable substances. Preferably, a pharmaceutical composition contains a probiotic bacterium and/or an active agent, in particular a cannabinoid and/or a curcuminoid, and a carrier, a solvent, and/or further additives such as buffers and preservatives. In particular embodiments, the therapeutic composition contains only water in addition to the specified substances probiotic bacterium, cannabinoid, and/or curcuminoid.

For purposes of the present invention, curcuminoids comprise, in particular, curcumin, demethoxycurcumin and bisdemethoxycurcumin, and mixtures thereof. In a particular embodiment, the curcuminoid is curcumin or a mixture containing curcumin. In a particular embodiment, the curcuminoid is a mixture containing curcumin, optionally demethoxycurcumin, and optionally bisdemethoxycurcumin. For example, the curcuminoid may be a mixture containing at least 60%, in particular at least 70% curcumin, at most 30%, in particular at most 20% demethoxycurcumin, and at most 10%, in particular at most 5% bisdemethoxycurcumin. Curcumin is also known as diferuloylmethane or (E,E)-1,7-bis-(4-hydroxy-3-methoxyphenyl)-1,6-heptadiene-3,5,-dione. The curcuminoid, in particular curcumin, can be obtained from a natural source such as the rhizome of the plant turmeric (Curcuma longa) or synthetically.

For purposes of the present invention, cannabinoids comprise, in particular, cannabidiol (CBD), delta-9-tetrahydrocannabinol (THC), cannabichromene, cannabielsoin, delta-8-tetrahydrocannabinol, iso-tetrahydrocannabinol, cannabicyclol, cannabicitran, and acids of these compounds such as cannabidiolic acid (CBDA) and delta-9-tetrahydrocannabinolic acid (THCA), and mixtures thereof. In a particular embodiment, the cannabinoid is CBD or a mixture containing CBD. In a further embodiment, the cannabinoid is THCA or a mixture containing THCA. In a further embodiment, the cannabinoid is THC or a mixture containing THC. The cannabinoid, particularly CBD, THC, and/or THCA, can be obtained from a natural source such as the hemp plant or synthetically.

In certain embodiments, the probiotic bacterium and the active agent or corresponding mixtures are used in the form of a therapeutic composition. In certain embodiments, the probiotic bacterium and the active agent are present together in a therapeutic composition.

The administration to the patient can be done by any suitable route of administration. In particular embodiments, the substances or mixtures of substances, in particular the therapeutic compositions, are administered orally. In particular, treatment or prevention is achieved by regular administration over an extended period of time, preferably continuously. In one embodiment, the substances are administered in a therapeutically effective amount.

Amyloidosis is a disease with accumulation of proteins. The enriched proteins form insoluble depositions (amyloid) such as fibrils. In particular embodiments, amyloidosis is a tauopathy. In tauopathies, tau protein is deposited in nerve and glial cells. In particular, the group of tauopathies comprises Alzheimer's disease, chronic traumatic encephalopathy (CTE), Parkinson's disease, corticobasal degeneration, and frontotemporal dementia. In a preferred embodiment, the amyloidosis is Alzheimer's disease. In Alzheimer's disease, plaques composed of misfolded beta-amyloid (Aβ) peptides form in the affected person's brain, and faulty tau proteins attach to axons from within the cell body.

Treatment is given to a patient in need thereof. These are in particular patients suffering from amyloidosis such as Alzheimer's disease. The treatment can be given at the onset of the disease as well as in advanced disease. Prevention can be done for any individual. In particular, prevention is applied to individuals of older age, such as from the age of 60, 65, 70 or 75. However, individuals of lesser ages may also benefit from prevention, such as individuals at least 20 years of age, at least 30 years of age, at least 40 years of age, or at least 50 years of age. In individuals with incipient or undiagnosed amyloidosis, prevention and treatment can be intertwined. The patients and individuals are in particular humans.

In certain embodiments, the probiotic bacterium and optionally the active agent are administered in one or more pharmaceutical compositions containing substantially only water in addition to these substances. In specific embodiments, the pharmaceutical compositions contain only water in addition to these substances. In certain embodiments, the pharmaceutical compositions contain these substances in high concentrations. For example, the concentration of curcuminoid, in particular curcumin, is at least 1 g/l, e.g., at least 2 g/l, at least 3 g/l, at least 5 g/l, at least 10 g/l, at least 15 g/l, or approximately 20 g/l. For example, the concentration of the cannabinoid, in particular CBD or THCA, may be at least 0.5 mg/l, preferably at least 1 mg/l, at least 10 mg/l, at least 100 mg/l, at least 250 mg/l, or at least 1 g/l.

The term “containing” or related expressions, where used herein, shall mean, in addition to the ordinary meaning thereof, “consisting essentially of” and “consisting of”. Thus, the term “containing” also refers to embodiments in which the subject matter “containing” certain listed elements does not contain further elements, as well as embodiments in which the subject matter “containing” certain listed elements additionally contains further elements. The term “consisting essentially of” relates in particular to embodiments in which the subject matter contains 10% or less, in particular 5% or less, additional elements in addition to the specifically listed elements of which the subject matter consists essentially.

### FIGURES

FIG. 1 shows the results of an Alzheimer's disease paralysis assay. A C. elegans nematode strain with inducible amyloid-β accumulation (CL4176) was exposed to a mixture of different probiotic bacteria alone or in combination with. The percentage of nematodes that have not become immobile 72 h after induction of Aβ accumulation is indicated. Untreated nematodes served as controls.

FIG. 2 shows the results of an Alzheimer's disease paralysis assay. A C. elegans nematode strain with inducible amyloid-β accumulation (CL4176) was exposed to a mixture of different probiotic bacteria at different concentrations. The percentage of nematodes that have not become immobile 96 h after induction of Aβ accumulation is indicated.

FIG. 3 shows the results of an Alzheimer's disease paralysis assay. A C. elegans nematode strain with inducible amyloid-β accumulation (CL4176) was exposed to various active agents that reduce the effects of Aβ accumulation without or together with the probiotic bacterium Lactobacillus rhamnosus. The percentage of nematodes that have not become immobile 96 h after induction of Aβ accumulation is indicated. Untreated (MQ) or ethanol-treated nematodes served as controls.

FIG. 4 shows the results of an Alzheimer's disease paralysis assay. A C. elegans nematode strain with inducible amyloid-β accumulation (CL4176) was exposed to various active agents that reduce the effects of Aβ accumulation without or together with the probiotic bacterium Lactobacillus plantarum. The percentage of nematodes that have not become immobile 96 h after induction of Aβ accumulation is indicated. Only nematodes treated with the solvent (water) served as controls.

FIG. 5 shows the results of an Alzheimer's disease paralysis assay. A C. elegans nematode strain with inducible amyloid-β accumulation (CL4176) was exposed to various active agents that reduce the effects of Aβ accumulation without or together with the probiotic bacterium Lactobacillus rhamnosus. The percentage of nematodes that have not become immobile 96 h after induction of Aβ accumulation is indicated. Only nematodes treated with the solvent (water) served as controls.

FIG. 6 shows the results of an Alzheimer's disease paralysis assay. A C. elegans nematode strain with inducible amyloid-β accumulation (CL4176) was exposed to various active agents that reduce the effects of Aβ accumulation without or together with the probiotic bacterium Lactobacillus plantarum. The percentage of nematodes that have not become immobile 96 h after induction of Aβ accumulation is indicated. Only nematodes treated with the solvent (water) served as controls.

FIG. 7 shows the results of an Alzheimer's disease paralysis assay. A C. elegans nematode strain with inducible amyloid-β accumulation (CL4176) was exposed to curcumin, the probiotic bacterium Pseudomonas fluorescens, or a combination thereof. The percentage of nematodes that have not become immobile 96 h after induction of Aβ accumulation is indicated. Only nematodes treated with the solvent (water) served as controls.

FIG. 8 shows the results of an Alzheimer's disease paralysis assay. A C. elegans nematode strain with inducible amyloid-β accumulation (CL4176) was exposed to CBD, the probiotic bacterium Pseudomonas fluorescens, or a combination thereof. The percentage of nematodes that have not become immobile 96 h after induction of Aβ accumulation is indicated. Only nematodes treated with the solvent (water) served as controls.

### EXAMPLES

### Example 1

### Therapeutic Effect of Probiotic Bacteria

The effect of probiotic bacteria and their combinations with other active agents has been studied in C. elegans nematode strains as an animal model for amyloidoses such as Alzheimer's disease. These strains accumulate amyloid-β peptides that form plaques in the nematodes and lead to paralysis of the animals. In certain strains, such as strain CL4176, Aβ accumulation occurs only in response to heat shock. Thus, the Alzheimer's disease pattern is inducible in these strains. The number of paralyzed nematodes after a certain period of time following induction of Aβ accumulation indicates the extent of Alzheimer's disease. Thus, the effect of certain substances to which the nematodes are exposed on the number of paralyzed animals is a measure of the anti-Alzheimer effect of these substances.

Nematodes of strain CL4176 were synchronized at the young larval stage and allowed to grow to the L4 stage on normal growth medium for 72 h at 16° C. The probiotic bacteria to be tested were added to the growth medium. Then the nematodes were exposed to the test substance and the temperature was increased to 25° C. This heat shock induced Aβ accumulation, simulating the onset of Alzheimer's disease. Approximately every 24 h, the number of animals that were still mobile and those that were paralyzed as a result of Aβ accumulation was determined.

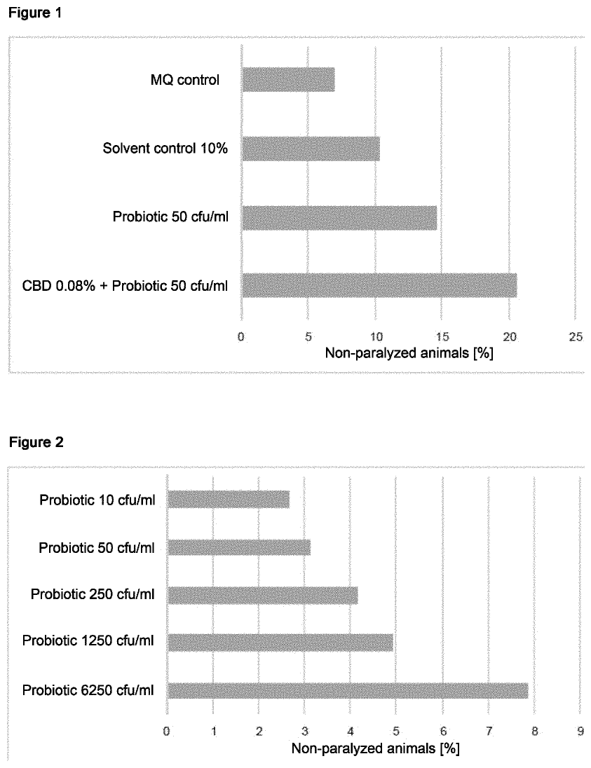

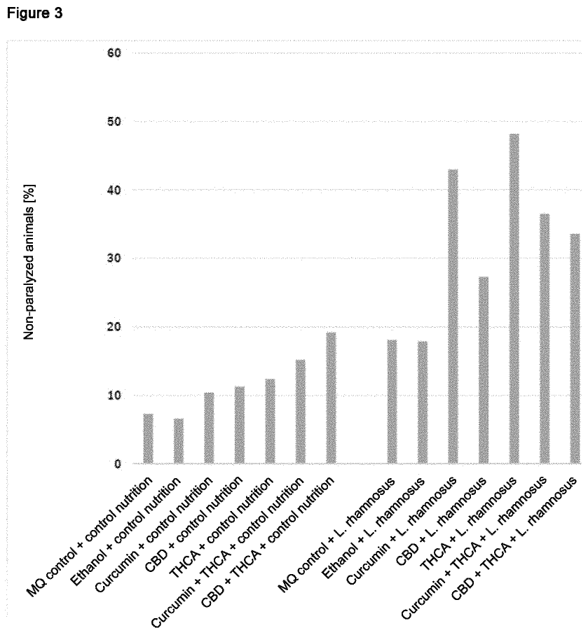

C. elegans of strain CL4176 were exposed to a mixture of different probiotic bacteria at a bacterial concentration of 50 cfu (colony-forming units) in this assay. The bacterial mixture contained the following bacterial species: Enterococcus faecium, Bifidobacterium longum, Streptococcus thermophilus, Lactobacillus acidophilus, Lactobacillus plantarum and Bifidobacterium breve. The probiotic bacterial mixture showed a therapeutic effect in this Alzheimer's disease assay 72 h after induction of Aβ accumulation. In another series of tests, the nematodes were additionally treated with cannabidiol (CBD). CBD significantly increased the effect of probiotic bacteria (see FIG. 1). The anti-Alzheimer's effect of probiotic bacteria also showed a clear concentration dependence (see FIG. 2).

Thus, probiotic bacteria protect against the effects of Aβ accumulation and help prevent and treat amyloidoses such as Alzheimer's disease.

### Example 2

### Probiotic Bacterial Species Lactobacillus rhamnosus and Lactobacillus plantarum

The two probiotic bacterial strains Lactobacillus rhamnosus and Lactobacillus plantarum were allowed to grow to stationary phase and mixed with the normal diet of nematodes (OP50) E. coli bacteria). C. elegans of strain CL4176 at L4 larval stage were exposed to the probiotic bacteria and other active agents in the induction of Aβ accumulation by increasing the temperature to 25° C. The number of paralyzed animals was checked every 24 h and determined after 96 h.

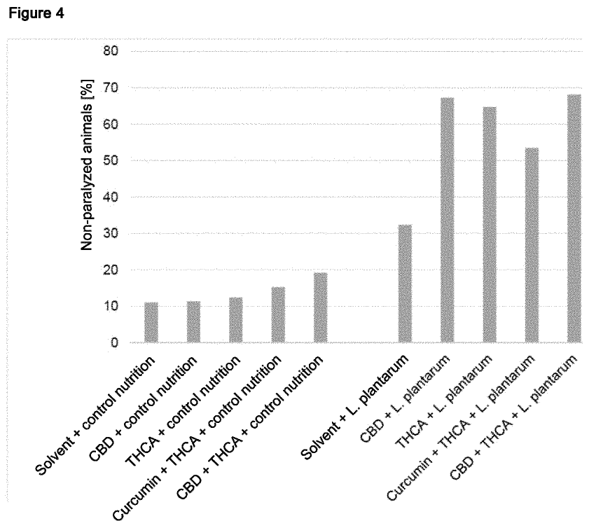

Both Lactobacillus rhamnosus and Lactobacillus plantarum showed a reduction in paralysis due to Aβ accumulation (see FIGS. 3 and 4). Similarly, the number of paralyzed nematodes was reduced when treated with curcumin (0.08% (v/v)), CBD (0.08% (v/v)), THCA (60 μl), or combinations thereof. The combination of Lactobacillus rhamnosus or Lactobacillus plantarum with these active agents showed significant to synergistic prevention of the effects of Aβ accumulation (see FIGS. 3 and 4).

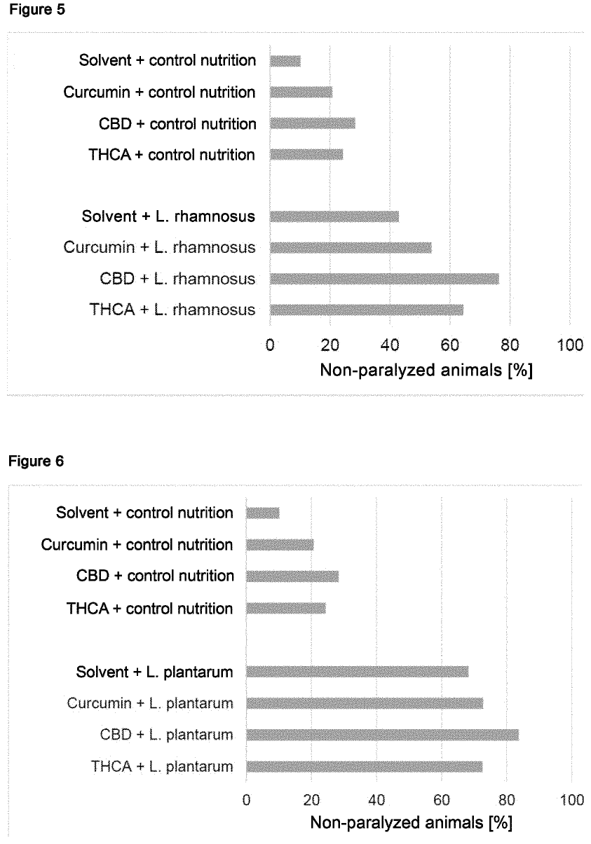

Corresponding results were also obtained when the probiotic bacterial strains were used in the growth phase, i.e., at a much lower concentration at an OD600 of 0.25 (see FIGS. 5 and 6).

The probiotic strains used thus showed a clear therapeutic effect against amyloidoses such as Alzheimer's disease, which could be further synergistically increased by combining them with further active agents such as curcumin, CBD and THCA.

### Example 3

### Probiotic Bacterial Species Pseudomonas fluorescens

In the described Alzheimer's disease animal model assay, the effect of the bacterial strain Pseudomonas fluorescens was tested. Upon induction of Aβ accumulation by increasing temperature to 25° C., nematodes were exposed to Pseudomonas fluorescens bacteria, curcumin or CBD, or a combination of the bacteria and the active agent. The number of paralyzed nematodes was determined 96 h after induction of Aβ accumulation.

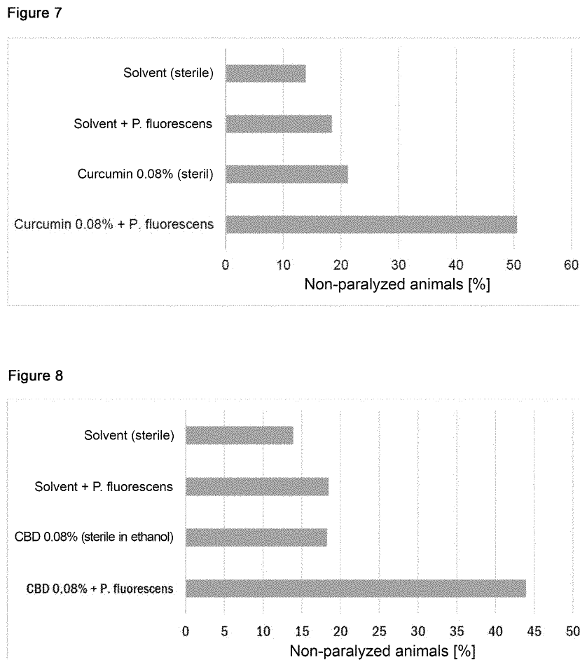

Pseudomonas fluorescens bacteria as well as curcumin and CBD individually showed a reduction in paralytic nematodes. A strong synergistic increase in effect was observed with the combination of Pseudomonas fluorescens and curcumin or CBD (see FIGS. 7 and 8).

The probiotic bacterial strain Pseudomonas fluorescens thus had a therapeutic effect against amyloidoses such as Alzheimer's disease, which could be synergistically increased when combined with active agents such as curcumin and CBD.

1. Use of a probiotic bacterium for the manufacture of a medicament for the treatment or prevention of amyloidosis.

2. Use in accordance with claim 1, wherein the probiotic bacterium is administered in combination with an active agent that is therapeutically effective in the treatment or prevention of amyloidosis.

3. Use in accordance with claim 2, wherein the active agent is selected from the group consisting of cannabinoids, curcuminoids, and mixtures thereof.

4. Use in accordance with claim 3, wherein the cannabinoid is a mixture containing cannabidiol and/or THCA.

5. Use in accordance with claim 3, wherein the cannabinoid is cannabidiol.

6. Use in accordance with claim 3, wherein the cannabinoid is THCA.

7. Use in accordance with any of claims 3 to 6, wherein the curcuminoid is a mixture containing curcumin.

8. Use in accordance with any of claims 3 to 6, wherein the curcuminoid is curcumin.

9. Use in accordance with any of claims 1 to 8, wherein the probiotic bacterium is selected from the group consisting of bacteria of the genus Lactobacillus, bacteria of the genus Streptococcus, bacteria of the genus Bifidobacterium, bacteria of the genus Enterococcus, and bacteria of the genus Pseudomonas.

10. Use in accordance with any of claims 1 to 8, wherein the probiotic bacterium is a lactic acid bacterium.

11. Use in accordance with any of claims 1 to 8, wherein the probiotic bacterium is selected from the group consisting of Enterococcus faecium, Bifidobacterium longum, Streptococcus thermophilus, Lactobacillus acidophilus, Lactobacillus plantarum, Lactobacillus rhamnosus, Bifidobacterium breve, and Pseudomonas fluorescens.

12. Use in accordance with any of claims 1 to 8, wherein the probiotic bacterium is Lactobacillus rhamnosus or Lactobacillus plantarum.

13. Use in accordance with any of claims 1 to 12, wherein the probiotic bacterium and optionally the further active agent are contained in a pharmaceutical composition.

14. Use in accordance with claim 13, wherein the pharmaceutical composition contains, in addition to the probiotic bacterium and optionally the further active agent, only water.

15. Use in accordance with any of claims 1 to 14, wherein the amyloidosis is Alzheimer's disease.
